(19) United States
(12) Patent Application Publication
(10) Pub. No.: US 2012/0252870 A1
(43) Pub. Date: Oct. 4, 2012

(54) Indian Hedgehog (Ihh) as a Marker to Predict Osteoarthritis (OA) and Methods for the Prevention and Treatment of OA

(75) Inventor: Lei Wei, Barrington, RI (US)

(73) Assignee: Rhode Island Hospital

(21) Appl. No.: 13/400,011

(22) Filed: Feb. 17, 2012

Related U.S. Application Data
(60) Provisional application No. 61/444,727, filed on Feb. 19, 2011.

(51) Int. Cl. G01N 33/566; A61K 31/713; A61K 31/58; A61P 19/02; A61K 31/352; A61K 31/343; A61K 31/27; G01N 33/577; C12Q 1/68; A61K 31/565

(52) U.S. Cl. 514/44 A; 435/6.12; 514/173; 514/182; 514/456; 514/462; 514/481; 435/7.1; 435/7.92

(57) ABSTRACT

Methods of diagnosing osteoarthritis are carried out by measuring levels of Ihh, and methods of treating osteoarthritis comprise inhibiting Ihh to reduce or prevent cartilage degradation.

### RELATED APPLICATION

This application claims priority to, and the benefit of, U.S. Patent Application No. 61/444,727, filed Feb. 19, 2011, which is incorporated herein by reference in its entirety.

### FIELD OF THE INVENTION

The field of the invention relates to arthritic conditions.

### BACKGROUND OF THE INVENTION

Osteoarthritis (OA) is a leading cause of disability with aging and affects over 27 million people in the United States but the pathogenesis still remains unknown. OA is characterized by progressive degeneration of the cartilage at the ends of the bones.

### SUMMARY OF THE INVENTION

Indian Hedgehog (Ihh), a bioactive protein found in articulating joints, is involved in cartilage degradation and subsequent development of OA. The data described herein demonstrates a dramatic increase in the Ihh expression in the joints of patients with OA. The amount of Ihh is correlated with the severity of cartilage damage in OA patients. The Ihh in the cartilage tissue culture induces the release of degradative enzymes (matrix metalloproteases), which in turn induces articular cartilage degeneration. These findings indicate that Ihh plays a critical role in OA cartilage degeneration. Ihh is therefore useful as a marker to predict OA cartilage damage, and targeting Ihh to reduce its expression or activity confers therapeutic benefit by preventing and treating OA.

Accordingly, a method of diagnosing or predicting the development of osteoarthritis, is carried out by detecting the presence of an Indian hedgehog (Ihh) gene product in a bodily fluid, wherein an increase level of the gene product compared to a normal level indicates a diagnosis of osteoarthritis or a prediction of development of osteoarthritis. The gene product comprises a polypeptide, protein, or transcript (e.g., mRNA). The bodily fluid comprises synovial fluid, blood, or serum. For example, synovial fluid is removed from an articulating joint such as a knee, elbow, or shoulder using arthroscopic techniques. An Ihh level that is at least 10%, 25%, 50%, 60%, 75%, 2-fold, 5-fold, 10-fold or greater than the level in a bodily fluid (e.g., synovial fluid) of a normal joint (i.e., one that is known not to be affected by cartilage degradation or osteoarthritis) indicate a condition of osteoarthritis or a pre-arthritic condition that is predicted to develop into clinical osteoarthritis in the future (e.g., 1, 2, 5, 10, or 20 years). In such as case, early therapeutic intervention is indicated to prevent or slow the progression of the disease.

In addition to the diagnostic value of measuring Ihh levels, the level of the gene product correlates with the severity of osteoarthritis. The stage or level of progression of the condition is determined by the level of Ihh, thereby providing the physician valuable information regarding most appropriate therapeutic intervention strategy.

Also within the invention is a kit comprising a reagent that detects Ihh protein (e.g., a ligand or Ihh-specific antibody) or a reagent that detects an Ihh transcript (e.g., oligonucleotide primer pairs) and instructions for comparing a patient sample-derived Ihh level with a normal control level to diagnose or prognose osteoarthritis.

Therapeutic applications of the invention include a method of inhibiting cartilage damage in a subject, comprising identifying an injured or diseased joint, and contacting a tissue of the joint with an inhibitor of an Ihh gene product or an inhibitor of expression of the gene product. In this case, the subject is one who is suspected of suffering from or at risk of developing osteoarthritis due to age, overuse, injury, or other compromise of joint integrity, or is suffering from or at risk of developing a condition characterized by cartilage degeneration, e.g., rheumatoid arthritis.

In either case, the joint tissue is contacted by injecting or infusing the inhibitor into a joint space of the joint, e.g., by injection or infusion of the inhibitor into a synovium of a joint. Inhibitors include siRNA, antisense nucleic acids, small molecules, antibodies or fragments thereof.

The compositions used in therapeutic applications or diagnostic applications are purified. A purified composition such as a protein or peptide (e.g., antibody, fusion protein) is at least 60%, by weight, free from proteins and naturally occurring organic molecules with which it is naturally associated. Preferably, the preparation is at least 75%, more preferably 90%, and most preferably at least 99%, by weight, the desired composition. A purified antibody may be obtained, for example, by affinity chromatography. By “substantially pure” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

Other features and advantages of the invention will be apparent from the following description of the preferred embodiments thereof, and from the claims. Publications, U.S. patents and applications, GENBANK/NCBI accession numbers, and all other references cited herein, are hereby incorporated by reference.

### BRIEF DESCRIPTION OF THE DRAWINGS

FIG. 1 is a series photographs showing increased Ihh expression in OA cartilage. Ihh was analyzed with immunohistochemistry in knee joint cartilage from OA knees (n=17) and resection specimens with normal cartilage (n=6). The expression of Ihh is significantly increased in OA cartilage. Increased staining for Ihh protein is associated with increased severity of OA cartilage damage as demonstrated by Safranin O staining. In contrast, Ihh staining was minimal in normal cartilage.

FIG. 2 is a series of graphs showing increased Ihh expression in OA synovial fluid. (A) Radiographs confirm cartilage damage and joint space narrowing in OA patient and no joint changes in the normal control. (B) Representative Western blot demonstrates a high level of Ihh in human OA synovial fluid compared to normal controls. (C) Densitometry showed that the mean Ihh concentration was increased by 57.2% in the OA group (n=32) relative to the control group (n=31) (p<0.02).

FIG. 3 is a series of graphs and bar graphs showing increased expression of Ihh, type X collagen, and MMP-13 in OA cartilage. (A) Representative histologic sections from OA cartilage and adjacent relatively normal cartilage are shown. (B) Expression of Ihh, and typical molecular markers of chondrocyte hypertrophy: type X collagen and MMP-13, were determined by real time PCR. Bar graphs show the averages with SD, N=3, *: p<0.05. (C) Representative immunohistochemistry for type X collagen and MMP-13 in OA cartilage and cartilage from a control patient without OA.

FIG. 4 is a series of graphs showing that Ihh expression is associated with cartilage damage and chondrocyte morphological changes. (A) An increase Ihh staining in OA cartilage correlates with OA grade and a larger cell size. (B) There was a significant correlation (p<0.0001) between the Ihh intensity of the chondrocytes and the Mankin score (R²=0.66) (B-a) and the cell size and the Mankin score (R²=0.51) (B-b). (C) Chondrocytes change in shape from spindle to spherical (decrease ratio of major: minor axis) with OA progression. Bar graphs show the averages of quantified data of Ihh intensity and cell size from two hundred and twenty-eight cells. *: Significant difference compared, grade I OA; ̂: compared to grade II OA, p<0.05.

FIG. 5 is series of bar graphs showing that Ihh induces chondrocyte hypertrophy and regulates type X collagen and MMPs in OA chondrocytes. Human OA chondrocytes were transfected with Ihh siRNA or treated with recombinant Ihh protein at 5.0 μg/ml. The mRNA levels of Ihh, type X collagen, and MMPs were determined by qPCR. The endogenous Ihh was knocked down 80% by Ihh SiRNA. Knockdown of Ihh inhibited the expression of type X collagen and MMP13. Ihh treatment increased the expression of type X collagen, and MMP-1, -3, and -13. Bar graphs show the means and SD of data from three independent experiments (chondrocytes from three different patients) using Real Time PCR. *: p<0.05 compared to Mock for siRNA or to control for Ihh protein. ̂: p<0.05 compared to SiIhh treatment.

FIG. 6 is a scheme of experimental design for the in vivo study.

FIG. 7 is a panel of graphs showing that deletion of Ihh attenuates OA cartilage degeneration.

FIG. 8 is a panel of graphs showing that deletion of Ihh attenuates OA damage (OARSI Score).

### DETAILED DESCRIPTION OF THE INVENTION

Ihh promotes chondrocyte hypertrophy and plays a role in cartilage degeneration in osteoarthritis (OA). The study described herein determined the correlation between Mankin grade of OA (van der Sluijs et al., 1992, J. Orthop. Res. 10:58-61) and Ihh expression as well as the effect of Ihh on human chondrocyte hypertrophy and expression of MMP13.

OA Cartilage and synovial fluid (SF) samples were obtained during total knee arthroplasty. Normal cartilage samples were obtained from tumor resections, and SF from healthy volunteers and the contralateral uninjured knee of patients undergoing anterior cruciate ligament reconstruction. Knee radiographs were obtained to confirm no abnormal changes in normal controls prior to obtaining the samples. Expression of Ihh in cartilage and synovial fluid was determined semi-quantitatively using immunohistochemistry and by western blot, respectively. Chondrocyte size was measured using image analysis. Ihh, type X collagen and MMP-13 mRNA were determined by real time PCR and protein expression of type X collagen and MMP-13 by immunohistochemistry.

Ihh expression was found to be increased in OA cartilage and synovial fluid compared to normal control samples. Increased expression of Ihh was associated with the severity of OA and chondrocyte hypertrophy in the OA cartilage. Exposure to exogenous Ihh induced type X collagen and MMP-13 expression. Conversely, knockdown of Ihh by siRNA had the opposite effect.

The results of the study indicate that Ihh expression correlates with OA progression and promotes changes in chondrocyte morphology and gene expression consistent with chondrocyte hypertrophy and cartilage degradation seen in OA cartilage. Thus, Ihh represents a therapeutic target to prevent OA progression.

### Osteoarthritis and Ihh Involvement in Pathogenesis

OA is characterized by destruction and loss of articular cartilage, thereby causing chronic joint pain and disability. Aging, trauma, excessive mechanical load, and genetics all are associated with the development of OA. However, the exact pathogenesis of the disease remains unknown.

Ihh, a key signaling molecule, is primarily synthesized and expressed in prehypertrophic chondrocytes during growth plate development. Ihh regulates chondrocyte hypertrophy and endochondral ossification. Genetic studies using gene knockout mice have demonstrated that activation of Ihh downstream signaling pathways results in a decrease in articular cartilage thickness and proteoglycan (PG) content while inhibiting Ihh signaling results in an increase of articular cartilage thickness and PG. Upregulation of hedgehog signaling (Hh) in postnatal cartilage promotes chondrocyte hypertrophy and cartilage degradation. Mechanical overload or injury is a mechanism of OA development, and the Ihh signaling pathway mediates the response of chondrocytes to mechanical stress. Ihh plays a critical role during OA cartilage degradation.

Prior to the invention, there was no direct genetic evidence linking Ihh to OA development by promoting chondrocyte hypertrophy. To determine whether Ihh plays a role in human OA cartilage degradation, human OA cartilage and synovial fluid were collected, and the mRNA and protein levels of Ihh in OA cartilage and synovial fluid were directly compared to adjacent relatively normal cartilage and also to normal control cartilage and synovial fluid samples (from tumor resections and the contralateral uninjured knee of patients undergoing anterior cruciate ligament reconstruction respectively) to determine if the Ihh expression pattern is altered in human OA cartilage. Furthermore, in order to establish a functional role for Ihh in OA chondrocyte hypertrophy, the hypertrophic markers type X collagen and MMP-13 were quantified after over-expressing or knocking-down Ihh in human OA chondrocytes. The data from these studies provided direct evidence that Ihh signaling promotes OA progression.

| Ihh sequence | |
|---|---|
| Homo sapiens Indian hedgehog (IHH), mRNA | |
| (SEQ ID NO: 1) | |
| NM_002181 2074 bp mRNA | |
| ORIGIN | |
| 1 | atcagcccac caggagacct cgcccgccgc tcccccgggc tccccggcca tgtctcccgc |
| 61 | ccggctccgg ccccgactgc acttctgcct ggtcctgttg ctgctgctgg tggtgccggc |
| 121 | ggcatggggc tgcgggccgg gtcgggtggt gggcagccgc cggcgaccgc cacgcaaact |
| 181 | cgtgccgctc gcctacaagc agttcagccc caatgtgccc gagaagaccc tgggcgccag |
| 241 | cggacgctat gaaggcaaga tcgctcgcag ctccgagcgc ttcaaggagc tcacccccaa |
| 301 | ttacaatcca gacatcatct tcaaggacga ggagaacaca ggcgccgacc gcctcatgac |
| 361 | ccagcgctgc aaggaccgcc tgaactcgct ggctatctcg gtgatgaacc agtggcccgg |
| 421 | tgtgaagctg cgggtgaccg agggctggga cgaggacggc caccactcag aggagtccct |
| 481 | gcattatgag ggccgcgcgg tggacatcac cacatcagac cgcgaccgca ataagtatgg |
| 541 | actgctggcg cgcttggcag tggaggccgg ctttgactgg gtgtattacg agtcaaaggc |
| 601 | ccacgtgcat tgctccgtca agtccgagca ctcggccgca gccaagacgg gcggctgctt |
| 661 | ccctgccgga gcccaggtac gcctggagag tggggcgcgt gtggccttgt cagccgtgag |
| 721 | gccgggagac cgtgtgctgg ccatggggga ggatgggagc cccaccttca gcgatgtgct |
| 781 | cattttcctg gaccgcgagc ctcacaggct gagagccttc caggtcatcg agactcagga |
| 841 | ccccccacgc cgcctggcac tcacacccgc tcacctgctc tttacggctg acaatcacac |
| 901 | ggagccggca gcccgcttcc gggccacatt tgccagccac gtgcagcctg gccagtacgt |
| 961 | gctggtggct ggggtgccag gcctgcagcc tgcccgcgtg gcagctgtct ctacacacgt |
| 1021 | ggccctcggg gcctacgccc cgctcacaaa gcatgggaca ctggtggtgg aggatgtggt |
| 1081 | ggcatcctgc ttcgcggccg tggctgacca ccacctggct cagttggcct tctggcccct |
| 1141 | gagactcttt cacagcttgg catggggcag ctggaccccg ggggagggtg tgcattggta |
| 1201 | cccccagctg ctctaccgcc tggggcgtct cctgctagaa gagggcagct tccacccact |
| 1261 | gggcatgtcc ggggcaggga gctgaaagga ctccaccgct gccctcctgg aactgctgta |
| 1321 | ctgggtccag aagcctctca gccaggaggg agctggccct ggaagggacc tgagctgggg |
| 1381 | gacactggct cctgccatct cctctgccat gaagatacac cattgagact tgactgggca |
| 1441 | acaccagcgt cccccacccc cgtcgtggtg tagtcataga gctgcaagct gagctggcga |
| 1501 | ggggatggtt gttgacccct ctctcctaga gaccttgagg ctggcacggc gactcccaac |
| 1561 | tcagcctgct ctcactacga gttttcatac tctgcctccc ccattgggga gggcccattc |
| 1621 | catccatctt aggccccttt gggtgggctt gcgcctcagt tgatgctgct aaattccctg |
| 1681 | ggagccagca tggatctggc tggacccgat gctgtccaga actgggaagg ccacaggggt |
| 1741 | ggggcagcca tcccggccat tctgaggtat gacattcctc cccggccaca ctcctcaaga |
| 1801 | cacatccaga gactgttgct gtctgtgggc agagttctgt gttctggcca atgtgaccgt |
| 1861 | agtgccgggg actgggggag gtgggttgga tgtgcttgcc acccccccgg ctaagctccc |
| 1921 | ccttctgctg aaccatgatc cccaccccct ccgccggtca gtctcccata ccttatttat |
| 1981 | tggagtggag ggggaagccc atgggagaat tttggggatg ttttggtctt ttcttccttt |
| 2041 | tgtaataaaa attatttaag ttgttagagc caaa |

Ihh protein sequence
(SEQ ID NO: 2)
MSPARLRPRLHFCLVLLLLLVVPAAWGCGPGRVVGSRRRPPRKL
VPLAYKQFSPNVPEKTLGASGRYEGKIARSSERFKELTPNYNPDIIFKDEENTGADRL
MTQRCKDRLNSLAISVMNQWPGVKLRVTEGWDEDGHHSEESLHYEGRAVDITTSDRDR
NKYGLLARLAVEAGFDWVYYESKAHVHCSVKSEHSAAAKTGGCFPAGAQVRLESGARV
ALSAVRPGDRVLAMGEDGSPTFSDVLIFLDREPHRLRAFQVIETQDPPRRLALTPAHL
LFTADNHTEPAARFRATFASHVQPGQYVLVAGVPGLQPARVAAVSTHVALGAYAPLTK
HGTLVVEDVVASCFAAVADHHLAQLAFWPLRLFHSLAWGSWTPGEGVHWYPQLLYRLG
RLLLEEGSFHPLGMSGAGS

### Measurement/Detection of Ihh

The measurement of levels or amounts of Ihh is determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, amounts of Ihh can be measured using reverse-transcription-based PCR assays (RT-PCR), e.g., using primers specific for the differentially expressed sequence of genes or by branch-chain RNA amplification and detection methods by Panomics, Inc. Amounts of Ihh can also be determined at the protein level, e.g., by measuring the levels of peptides encoded by the gene products described herein, or subcellular localization or activities thereof using technological platform such as for example AQUA® (HistoRx, New Haven, Conn.) or U.S. Pat. No. 7,219,016. Such methods are well known in the art and include, e.g., immunoassays based on antibodies to proteins encoded by the genes, aptamers or molecular imprints. Any biological sample can be used for the detection/quantification of Ihh nucleic acid or protein or its activity, e.g., a bodily fluid such as synovial fluid is used for the detection/measurement. In preferred embodiments, the bodily fluid includes synovial fluid, blood, or serum. Alternatively, a suitable method can be selected to determine the activity of proteins encoded by the marker genes according to the activity of each protein analyzed.

The Ihh proteins or polypeptides thereof can be detected in any suitable manner, but is typically detected by contacting a sample from the subject with an antibody which binds the Ihh protein or polypeptide and then detecting the presence or absence of a reaction product. The antibody may be monoclonal, polyclonal, chimeric, or an antibody fragment. The step of detecting the reaction product may be carried out with any suitable immunoassay.

Immunoassays carried out in accordance with the present invention may be homogeneous assays or heterogeneous assays. In a homogeneous assay the immunological reaction usually involves the specific antibody (e.g., anti-Ihh protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

Using sequence information provided by the database entries for the Ihh sequences, expression of the Ihh sequences can be detected (if present) and measured using techniques well known to one of ordinary skill in the art. For example, sequences within the sequence database entries corresponding to Ihh sequences, or within the sequences disclosed herein, can be used to construct probes for detecting Ihh RNA sequences in, e.g., Northern blot hybridization analyses or methods which specifically, and, preferably, quantitatively amplify specific nucleic acid sequences. As another example, the sequences can be used to construct primers for specifically amplifying the Ihh sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), e.g., using primers specific for the differentially expressed sequences. RNA can also be quantified using, for example, other target amplification methods (e.g., TMA, SDA, NASBA), or signal amplification methods (e.g., bDNA), and the like.

### Diagnostic and Prognostic Methods

The invention allows the diagnosis and prognosis of osteoarthritis (OA). The risk of developing OA can be detected by measuring Ihh protein, polypeptide, or nucleic acids in a test sample (e.g., a subject derived sample), and comparing the measured amounts to reference or index values. Subjects identified as having an increased risk of OA are selected to be a candidate to receive appropriate treatment regimens.

The amount of the Ihh polypeptides, protein, or nucleic acid can be measured in a test sample and compared to the “normal control level,” utilizing techniques such as reference limits, discrimination limits, or risk defining thresholds to define cutoff points and abnormal values. The “normal control level” means the level of Ihh typically found in a subject not suffering from OA. Alternatively, the normal control level can be a database of Ihh levels from previously tested subjects who did not develop OA over a clinically relevant time horizon. Identifying the subject at risk of having OA enables the selection and initiation of various therapeutic interventions or treatment regimens in order to delay, reduce or prevent that subject's developing of OA. An Ihh level that is at least 10%, 25%, 50%, 60%, 75%, 2-fold, 5-fold, 10-fold or greater than the level in a bodily fluid (e.g., synovial fluid) of a normal joint (i.e., one that is known not to be affected by cartilage degradation or osteoarthritis) indicate a condition of osteoarthritis or a pre-arthritic condition that is predicted to develop into clinical osteoarthritis in the future (e.g., 1, 2, 5, 10, or 20 years).

Levels of Ihh polypeptide, protein, or nucleic acid also allows for the course of treatment of disease to be monitored. In this method, a biological sample can be provided from a subject undergoing treatment regimens, e.g., drug treatments, for OA. If desired, biological samples are obtained from the subject at various time points before, during, or after treatment.

Also provided are methods for determining the severity/aggressiveness of OA by examining the presence (e.g, expression) or absence of an Ihh nucleic acid, polypeptide or activity in a test sample (i.e., a patient derived sample). Preferably, the test sample is a bodily fluid, such as synovial fluid, blood, or serum. An increase in the level of the Ihh nucleic acid, polypeptide or activity compared to a control sample is indicative of the severity of the OA in the subject. By normal control level is meant the expression level of an Ihh nucleic acid, polypeptide or activity typically found in a subject not suffering from OA. The alteration in the amount of the Ihh nucleic acid, polypeptide or activity is statistically significant. By statistically significant is meant that the alteration is greater than what might be expected to happen by change alone. Statistical significance is determined by method known in the art. For example statistical significance is determined by p-value. The p-value is a measure of probability that a difference between groups during an experiment happened by chance. (P(z≧z_{observed})). For example, a p-value of 0.01 means that there is a 1 in 100 chance the result occurred by chance. The lower the p-value, the more likely it is that the difference between groups was caused by treatment. An alteration is statistically significant if the p-value is at least 0.05. Preferably, the p-value is 0.04, 0.03, 0.02, 0.01, 0.005, 0.001 or less.

The subject is preferably a mammal. The mammal is, e.g., a human, non-human primate, mouse, rat, dog, cat, horse, or cow. Subjects are typically human females or human males.

### Methods of Treating or Preventing OA

The invention provides a method for treating, preventing or alleviating a symptom of OA, such as cartilage damage or associated articulating joint pain, in a subject by decreasing expression or activity of Ihh. Therapeutic compounds are administered prophylactically or therapeutically to subject suffering from or at risk of (or susceptible to) developing OA. Such subjects are identified using standard clinical methods or by detecting an aberrant level of expression or activity of an Ihh protein, polypeptide or nucleic acid.

The method also includes decreasing the expression, or function, or both, of one or more gene products of Ihh whose expression is aberrantly increased (“overexpressed gene”) in OA subject relative to normal subject. Expression is inhibited in any of several ways known in the art. For example, expression is inhibited by administering to the subject a nucleic acid that inhibits, or antagonizes, the expression of the overexpressed gene or genes, e.g., an antisense oligonucleotide or a siRNA which disrupts expression of the overexpressed gene or genes. Exemplary siRNA against Ihh can be found at Santa Cruz Biotechnology, Inc (Catalog Nos: sc-37207 and sc-37206).

Alternatively, function of one or more gene products of the overexpressed Ihh is inhibited by administering a compound that binds to or otherwise inhibits the function of the gene products. For example, the compound is an antibody which binds to the overexpressed gene product or gene products. The compound can also be a small molecule. Examples of small molecules that inhibit hedgehog signaling are:

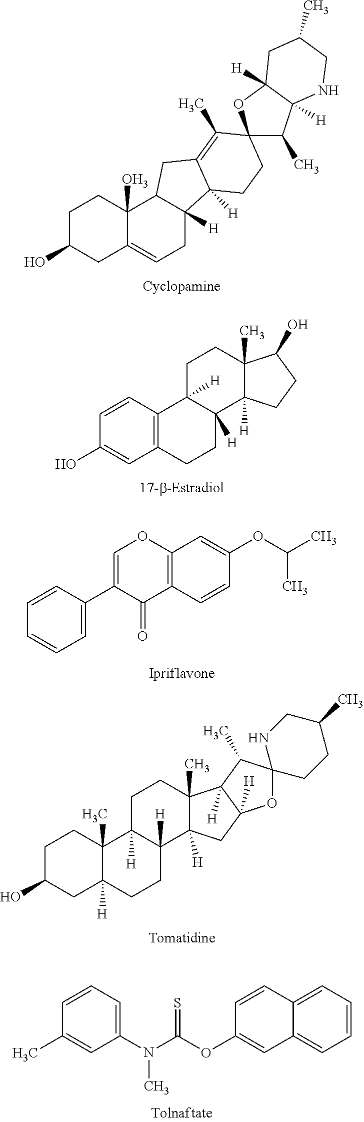

(Lipinski et al. Toxicology in Vitro, 24, 1404-1409, 2010 and Bassleer et al. Osteoarthritis and Cartilage, 4, 1-8, 1996, hereby incorporated by reference.)

These modulatory methods are performed ex vivo or in vitro (e.g., by culturing the cell with the agent) or, alternatively, in vivo (e.g., by administering the agent to a subject). The method involves administering a protein or combination of proteins or a nucleic acid molecule or combination of nucleic acid, molecules as therapy to counteract aberrant expression or activity of Ihh.

### Therapeutic Administration

An inhibitor of Ihh is formulated to be compatible with its intended route of administration. Inhibitors of Ihh are preferably administered to a mammal via intra-articular, or subcutaneous injection or infusion, or implant). Articulating joints, e.g., knee, elbow, shoulder, or hip are treated in this manner. Preferably, the inhibitor is administered locally, for example, directly to the joint where repair or prevention is needed. Such methods are known in the art, e.g., as described in Wen et al., 2000, Am Fam Physician. 62:565-570 or Lockman et al., 2006, Can Fam Physician, 52: 1403-1404. Therapeutic compositions are administered in a pharmaceutically acceptable carrier (e.g., physiological saline). Carriers are selected on the basis of mode and route of administration and standard pharmaceutical practice. A therapeutically effective amount of a therapeutic composition (e.g., lubricating polypeptide) is an amount which is capable of producing a medically desirable result, e.g., reduction in cartilage degradation of or in a mammalian joint, in a treated animal. A medically desirable result is a reduction in pain (measured, e.g., using a visual analog pain scale described in Peyron et al., 1993, J. Rheumatol. 20 (suppl. 39):10-15) or increased ability to move the joint (measured, e.g., using pedometry as described in Belcher et al., 1997, J. Orthop. Trauma 11:106-109).

Other examples of routes of administration include parenteral, e.g., intravenous, intradermal, subcutaneous, oral (e.g., inhalation), transdermal (i.e., topical) or local injection or infusion. Solutions or suspensions used for parenteral, intradermal, subcutaneous, local injection or infusion application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. For example an inhibitor of the present invention is administered intraoperatively, arthroscopically, or by local direct injection into the affected tissue, such as a synovium of a joint.

Examples of dosing regimens that can be used in the methods of the invention include, but are not limited to, daily, three times weekly (intermittent), weekly, or every 14 days. In certain embodiments, dosing regimens include, but are not limited to, monthly dosing or dosing every 6-8 weeks. Typically, 1-3 injunctions are administered per year per articulating joint.

### Kits

The invention also includes an Ihh-detection reagent, e.g., nucleic acids that specifically identify an Ihh nucleic acid by having homologous nucleic acid sequences, such as oligonucleotide sequences, complementary to a portion of the Ihh nucleic acid or antibodies to proteins encoded by the Ihh nucleic acids packaged together in the form of a kit. The oligonucleotides can be fragments of the Ihh gene. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a Northern hybridization or a sandwich ELISA as known in the art.

For example, Ihh detection reagents can be immobilized on a solid matrix such as a porous strip to form at least one Ihh detection site. The measurement or detection region of the porous strip may include a plurality of sites containing a nucleic acid. A test strip may also contain sites for negative and/or positive controls. Alternatively, control sites cart be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of Ihh present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences fragment of Ihh. In various embodiments, the expression of 2, 3, 4, 5, 6, 7, 8, 9, 10, 15, 20, 25, 40, 50, or more of the fragments of Ihh can be identified by virtue of binding to the array. The substrate array can be on, e.g., a solid substrate, e.g., a “chip” as described in U.S. Pat. No. 5,744,305. Alternatively, the substrate array can be a solution array. The skilled artisan can routinely make antibodies, nucleic acid probes, e.g., oligonucleotides, aptamers, siRNAs, antisense oligonucleotides, against Ihh.

The following materials and methods were used to generate the data described below.

### Specimens

Articular cartilage samples were obtained from patients with OA at time of total joint arthroplasty (N=17, 11 female, 6 male, mean±SD age 68.6±8.6, range 55-79). Normal control samples of articular cartilage were obtained from patients undergoing tumor resections (N=6, 6 male, mean±SD age 23.8±13.6, range 15-51). OA synovial fluid was also obtained during knee joint arthroplasty (N=32, 20 female, 12 male, mean±SD age 69.6±9.0, range 42-86). Normal synovial fluid samples were also collected from the contralateral uninjured knee of patients undergoing unilateral ACL reconstruction (ACLR) (n=30) and one healthy volunteer (N=31, male 17, female 14, mean±SD age 25.1±10.5, range 15-54). Inclusion criteria for using the contralateral knee of ACLR patients as a normal control were the absence of prior knee injury and normal standing radiographs.

### Synovial Fluid Analysis

Synovial fluid samples were centrifuged at 2,000 g for 10 minutes to remove cells and debris. The synovial fluid was aliquoted and frozen at −80° C. until analysis. Before performing the experiments, the synovial fluid samples were treated with 15 U/ml of bovine testicular hyaluronidase (HA) (Sigma Chemical Company, St Louis, Mo.) for 15 minutes at 37° C. to reduce viscosity and diluted with cell lysis buffer containing proteinase inhibitor (1:10, Roche Diagnostics, Indianapolis, Ind.).

### Chondrocyte Isolation and Primary Culture

Cartilage slices were removed from the tibia plateau and washed in Dulbecco's modified Eagle's medium (DMEM). Chondrocytes were isolated from cartilage using known methods. Small pieces of cartilage were minced with a scalpel and digested with pronase (Boehringer Roche) (2 mg/ml) in HBSS for 30 min at 37° C. with shaking. After digestion, the solution was removed, tissue pieces were washed once with DMEM, and digested with crude bacterial collagenase (Type IA, Sigma, C 2674) (1 mg/ml) for 6-8 h at 37° C. with shaking. Enzymatic digestion was stopped by adding DMEM containing 10% FBS. Residual multicellular aggregates were removed by filtering and the cells were washed 3 times with DMEM. Chondrocytes were incubated in DMEM containing 10% FCS, L-glutamine, and antibiotics, and allowed to attach to the surface of the culture dishes. Second passage human OA chondrocytes were transfected with Ihh siRNA or exposed to recombinant human Ihh protein (1705-HH, R&D systems, Inc. Minneapolis, Minn.) at different concentrations.

### Histology

Full thickness 2×2 cm cartilage samples were taken from each knee joint with a scalpel. Approximately half of the section (0.4-0.5 g) was used for RNA isolation and the remainder was fixed in 10% formalin for 72 hrs. The specimens were decalcified in Richman-Gelfand-Hill solution, processed in a Tissue-Tek VIP 1000 tissue processor (Model #4617, Miles, Elkhart, Ind.), and embedded in a single block of Paraplast X-tra (Fisher Scientific). Blocks were trimmed to expose tissue using a rotary microtome (Model #2030, Reichart-Jung, Wien, Austria). The slices were then cut into 6-μm sections and mounted on slides. Safranin-O staining was performed and the severity of cartilage damage was then assessed using the modified Mankin grading system. Grade I (Mankin score 0-2) represents minimal cartilage damage, while Grade II (Mankin score 3-10) and Grade III (Mankin score 11-18) represent more severe cartilage damage.

### Immunohistochemistry

To detect the distribution of Type X collagen and MMP13 in cartilage, 6 μm sections were collected on positively charged glass slides (Superfrost Plus, Fisher Scientific). The sections were dried on a hot plate to increase adherence to the slides. Immunohistochemistry was carried out using the Histostain-SP Kits (Zymed, Cat #95-9943). Sections were de-paraffined and rehydrated using conventional methods. Endogenous peroxidase was blocked by treating the sections with 3% hydrogen peroxide in methanol for 30 min. The sections were digested by 5 mg/mL hyaluronidase in PBS (Cat #H3506, Sigma-Aldrich, St. Louis, Mo.) for 20 min. Nonspecific protein binding was blocked by incubation with a serum blocking solution. The sections were incubated with affinity-isolated Ig G fractions of Ab against human Col X (2 μg/mL; Cat #234196 EMD Chemicals Inc., Gibbstown, N.J.) and an Antibody against MMP13 (2 μg/mL; Cat #sc-12363 Santa Cruz Biotechnology, Inc., Santa Cruz, Calif.) respectively at 4° C. overnight. The negative control sections were incubated with isotype control (2 μg/mL; Cat # MAB002 R&D Systems, Inc., Minneapolis, Minn.) in PBS. Thereafter, the sections were treated sequentially with biotinylated secondary antibody and streptavidin-peroxidase conjugate (Zymed), and then were developed in DAB chromogen (Zymed, Calif.). The sections were counterstained with hematoxylin (Zymed). Photography was performed with a Nikon E800 microscope (Melville, N.Y.).

To detect the distribution of Ihh in the cartilage, 6 μm cartilage sections were analyzed by immunofluorescent staining with a polyclonal Ab against Ihh (sc-1196; Santa Cruz Biotechnology, Inc, Santa Cruz Calif.). The sections were incubated with primary antibody at 4° C. overnight. After washing with PBS, affinity-purified TRITC conjugated donkey anti-goat secondary antibody (1:500, Jackson ImmunoResearch, West Grove, Pa.) was applied with Hoechst nuclear dye (0.5 mg/ml). The sections were washed and mounted in GEL/MOUNT™ (Cat. No: M01, Biomeda Corp. Foster city, CA). Single or multiple exposure photography was performed with a Nikon E800 microscope (Melville, N.Y.). The intensity of Ihh expression and the size of chondrocytes were visualized under fluorescence microscopy and quantified using Ivision software (4.0.10, BioVision Techologies, Exton, Pa.). The intensity of Ihh is a pixel unit of gray scale.

### Western Blot

Total protein of synovial fluid was quantified using the BAC Protein Assay Reagent Kit (Pierce, Rockford. IL). 14 μg of total protein was electrophoresed in 10% SDS PAGE under reducing conditions. After electrophoresis, proteins were transferred onto Immobilon-PVDF membrane (NJ0HYB0010, Fisher Scientific) and probed with a monoclonal antibody against Ihh (Cat #ab52919, abcam, Inc. Cambridge, Mass.). The antibody was diluted 1:1,000 in PBS-T containing 1% bovine serum albumin. Horseradish peroxidase-conjugated goat anti-rabbit IgG (H+L) (Bio-Rad Laboratories, Richmond, Calif.) was diluted 1:3000 in PBS-T and used as the secondary antibody. Visualization of immunoreactive proteins was achieved by using the ECL Western blotting detection reagents (Amersham, Arlington Heights, Ill.) and by subsequently exposing the membrane to Kodak X-Omat AR film. Band densities were quantified using Image Acquisition and Analysis Software (UVP Biosystems, Upland, Calif.).

Real Time RT-PCR (qPCR)

Total RNA was isolated from OA cartilage and the adjacent “relatively normal” cartilage from the opposite compartment (usually lateral) in the same specimen (Mankin scores: 0 or 1) or from chondrocytes incubated with Ihh protein or transfected with Ihh siRNA, using RNeasy isolation kit (Qiagen). 1 μg of RNA was reverse-transcribed to obtain first-strand cDNA using the iScript™ cDNA synthesis Kit (Bio-Rad, Hercules, Calif.). The quantification of mRNA for Ihh, type X collagen, and MMP-13 were determined using the QuantiTect SYBR Green PCR kit (QIAGEN, Valencia, Calif.) with the DNA Engine Opticon 2 Continuous Fluorescence Detection System (MJ Research, Waltham, Mass.). Each reaction was performed in triplicate. Ihh primers were as follows: forward, 5′-CATTGAGACTTGACTGGGCAAC-3′, and reverse, 5′-AGAGCAGGCTGAGTTGGGAGTCGC-3′. Type X collagen primers were as follows: forward, 5′-TGC CTC TTG TCA GTG CTA ACC-3′, and reverse, 5′-GCG TGC CGT TCT TAT ACA GG-3′. MMP-13 primers were as follows: forward, 5′-TGC TGC ATT CTC CTT CAG GA-3′, and reverse, 5′-ATG CAT CCA GGG GTC CTG GC-3′. MMP-1 primers were as follows: forward, 5′-CTG TTC AGG GAC AGA ATG TGC T-3′, and reverse, 5′-TTG GAC TCA CAC CAT GTG TT′. MMP-3 primers were as follows: forward, 5′-CCC TCC AGA ACC TGG GAC-3′, and reverse, 5′-ATA AAA GAA CCC AAA TTC TFC AAA A′.

Amplification conditions were as follows: 2 min of preincubation at 50° C., 10 min at 95° C. for enzyme activation, and 40 cycles at 95° C. denaturation for 10 sec, 55° C. annealing for 30 sec, and 72° C. extension for 30 sec. The cycle threshold (Ct) values for 18s RNA and that of samples were measured and calculated by computer software (MJ Research, Waltham, Mass.). Relative transcription levels were calculated as x=2^{-ΔΔCt}, in which ΔΔCt=ΔE−ΔC, and ΔE=CtₑₓₚCt₁₈ₛ; ΔC=Ct_{ctl}−Ct₁₈ₛ,

### Statistical Analysis

Two-tailed t-tests were used to compare mRNA levels from OA cartilage to its adjacent relatively normal cartilage. The mRNA levels in chondrocytes under different conditions were analyzed by one-way analysis of variance (ANOVA) with multiple pairwise comparisons made by the Student-Newman-Keuls method (3 comparisons or more) at a rejection level of 5% unless otherwise noted.

### Increased Ihh Expression in OA Cartilage

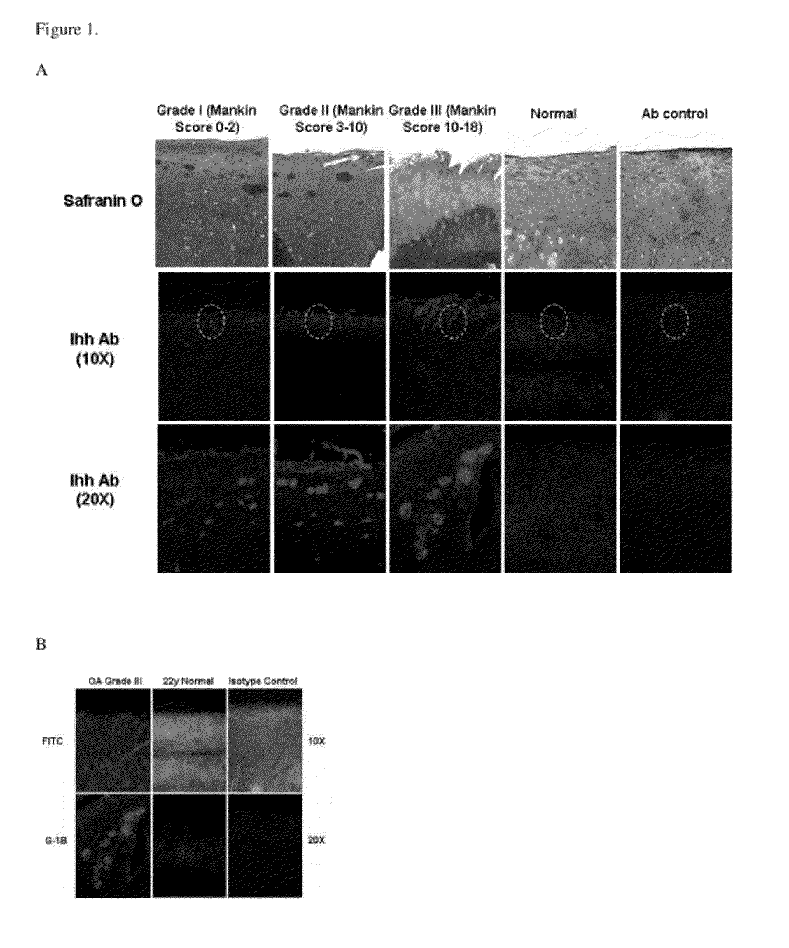

Knee joint cartilage samples from OA patients (OA) and patients undergoing tumor resections (normal controls) were obtained. The normal cartilage was collected from the central region of cartilage (thickest region). Immunofluorescent staining showed that the expression of Ihh protein was low in normal articular cartilage, but significantly greater in the OA cartilage samples (FIG. 1). The more intense staining was mainly found in the upper clusters of cells in OA cartilage, and the intensity of Ihh staining was associated with the severity of OA cartilage damage as determined by modified Mankin score (r2=0.6621). When compared to OA cartilage, no obvious staining of Ihh was observed in the normal cartilage.

### Increased Ihh Levels in OA Synovial Fluid

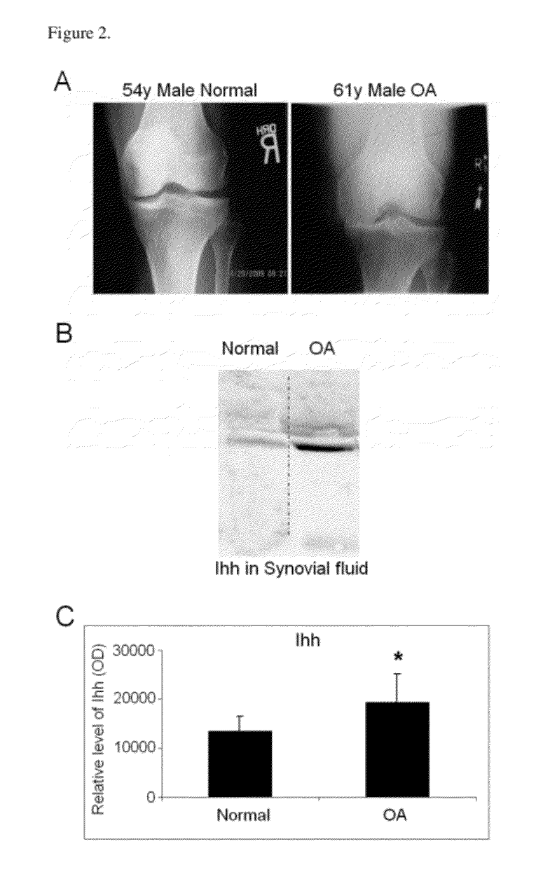

Synovial fluid samples collected from OA patients, age-matched normal controls, and the contralateral side of patients undergoing unilateral ACL reconstruction were processed for Ihh level by western blot. Ihh concentration was 57.2% higher in the synovial fluid of the OA patients when compared to that of the normal controls (FIG. 2).

### Increased Ihh, Type X Collagen, and MMP-13 Expression in OA Cartilage

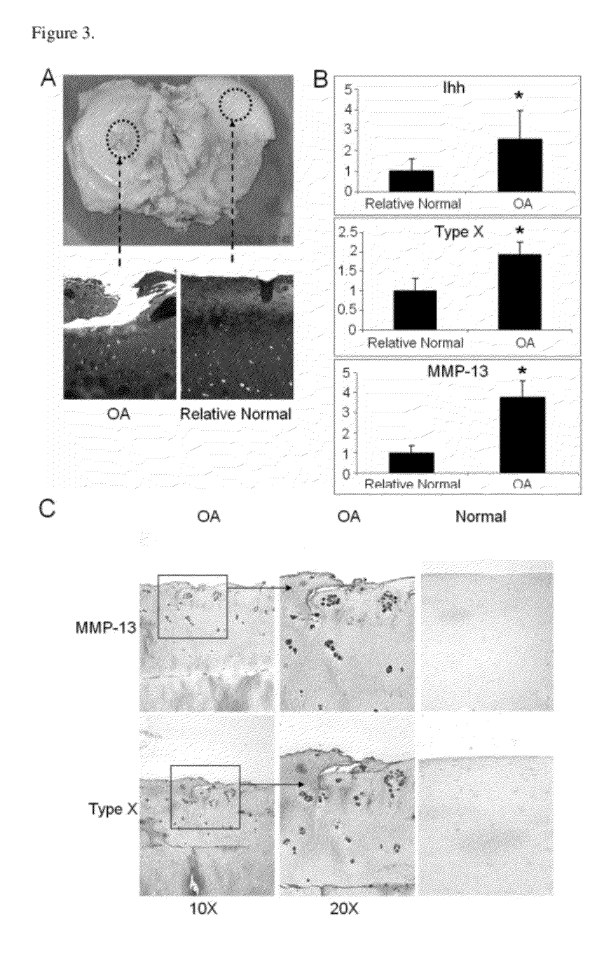

Total RNA was isolated from the cartilage samples. To reduce the influence of age and sex, the changes in gene expression were compared between cartilage samples obtained from the same patient. Real time PCR indicates that the levels of Ihh, type X collagen, and MMP-13 expression were increased in OA cartilage samples compared to the adjacent, relatively normal cartilage (FIG. 3A, B). Immunostaining showed increased type X collagen and MMP-13 in OA cartilage compared to cartilage from a normal patient without OA (FIG. 3C).

Increased Ihh Levels in OA Cartilage Associated with Altered Cell Morphology

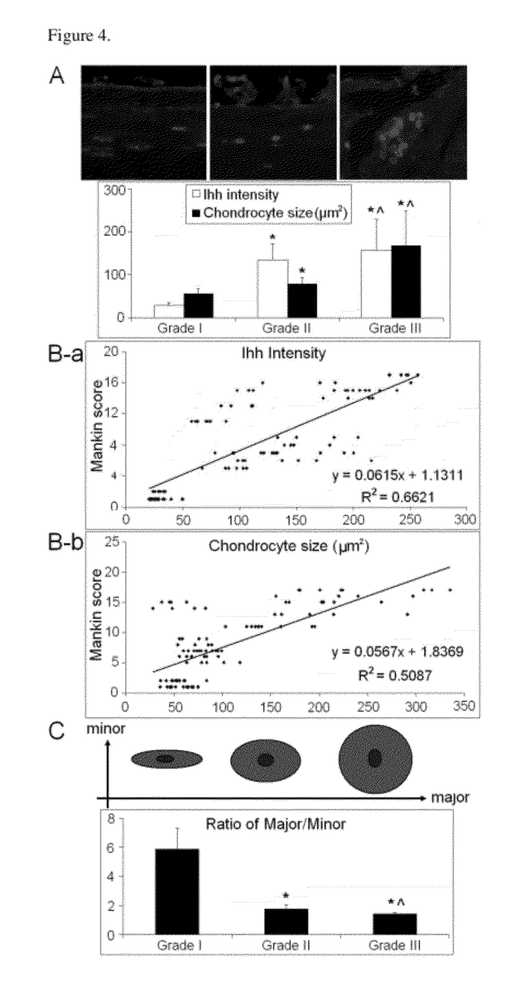

Increased Ihh staining was associated with the larger size of chondrocytes observed in OA cartilage and with increased cartilage damage (FIG. 4A). There was a significant correlation (p<0.0001) between the Ihh intensity of the chondrocytes and the Mankin score (r2=0.66) (FIG. 4B-a). In addition, chondrocytes became more spherical with increasing severity of OA. There was a significant correlation (p<0.0001) between the cell size and the Mankin score (r2=0.51) (FIG. 4B-b), analogous to the changes seen in chondrocyte morphology in the growth plate as chondrocytes become hypertrophic (FIG. 4C).

### Ihh Induces Hypertrophy in a Dose-Dependent Manner and Regulates Expression of Type X Collagen and MMPs in OA Chondrocytes

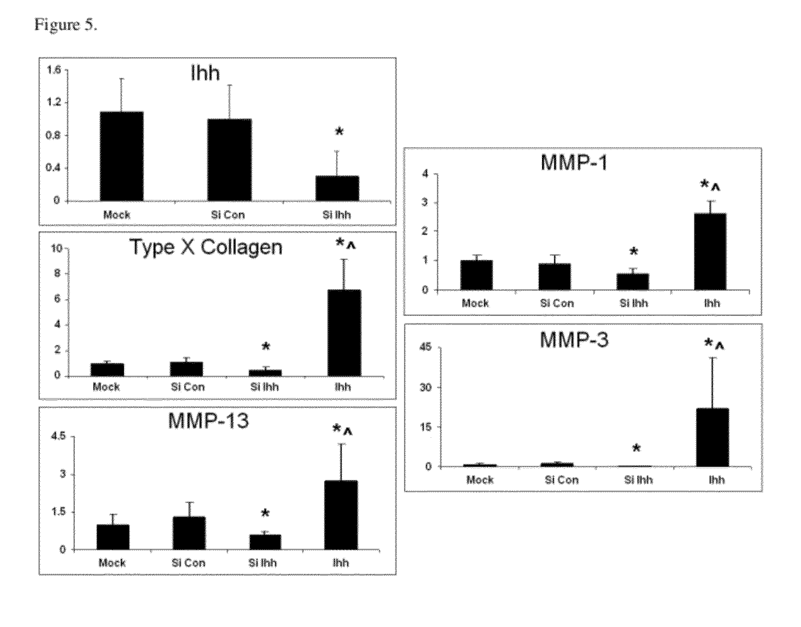

Exposure of OA chondrocytes to recombinant Ihh protein increased the expression of type X collagen, MMP-1, MMP-3, and MMP-13 mRNA. In contrast, knockdown of Ihh by transfecting chondrocytes with Ihh siRNA inhibited the expression of Ihh, type X collagen, and MMPs (FIG. 5).

### Activation of Indian Hedgehog Promotes Chondrocyte Hypertrophy and Upregulation of MMP-13 in Human Osteoarthritic Cartilage

Genetic data suggest that activating Hh downstream signaling using Ptch1 C/C, col2al-CreER mice causes OA, very early focal cartilage degradation in human knee articular cartilage is accompanied by up-regulation of collagenase activity and expression of genes associated with chondrocyte terminal differentiation and matrix degradation. However prior to the invention, it was not clear if increased Ihh signaling initiates OA or causes ongoing expression of genes known to be associated with OA development and cartilage degradation. It was also not known how the expression of Ihh varies with grade of OA.

The data described herein indicates that increased Ihh expression plays a critical role in human OA cartilage degradation. Cartilage and synovial fluid samples from patients with OA have elevated levels of Ihh when compared to normal control samples, and that the amount of Ihh was associated with severity of OA (FIGS. 1 and 2) and hypertrophic chondrocyte phenotype (FIG. 4). OA chondrocytes had more of a spherical cell shape and larger cell size than chondrocytes from normal cartilage (FIG. 4). The data show that upregulated Ihh promotes the hypertrophic phenotype and regulates typical hypertrophic markers such as type X collagen and MMP-13 (FIG. 5). Therefore up-regulation of Ihh signaling facilitates OA development by inducing chondrocyte hypertrophy and expression of genes known to cause cartilage degeneration. This result is consistent with the finding that type X collagen and MMP-13 are increased in OA cartilage.

OA articular chondrocytes recapitulate some of the differentiation processes that occur during fetal development. In the growth plate, Ihh is specifically expressed by the prehypertrophic chondrocytes. Ihh signaling mediates chondrocyte hypertrophy and endochondral bone formation, which are associated with MMP induced matrix degradation.

In addition to increased Ihh in OA tissues, the increase of Ihh is correlated with the severity of OA cartilage damage (FIG. 1), indicating that upregulated Ihh signaling plays a critical role in OA progression. The data show that high levels of Ihh are accompanied by increased cell size, altered cell morphology, and up-regulated hypertrophic markers type X collagen and MMP-13 (FIGS. 3 and 4), indicating that upregulated Ihh signaling regulates the chondrocyte hypertrophic phenotype and cartilage matrix degradation. Increased cell size (FIG. 4A) and increased ratio of minor axis to major axis along with OA progression (FIG. 4B) suggest that OA chondrocytes are of larger volume and more spherical cell shape. In articular cartilage, chondrocytes in the superficial zone are usually spindle-shaped, while chondrocytes in the deeper zone are more spherical, which are similar to hypertrophic cells. Thus, the finding that OA chondrocytes have more spherical cell morphology indicates that OA chondrocytes enter the hypertrophic stage induced by Ihh. Alternatively, the increasing cartilage damage in advanced OA leads to severe disruption or complete loss of the superficial zone, and thus may expose more spherical chondrocytes originally located in the deeper zones. In either case, these spherical chondrocytes undergo clustering in OA, a phenomenon not seen in normal articular cartilage.

The cartilage and synovial samples from the normal and OA patients revealed that cartilage degeneration is accompanied by enhanced Ihh synthesis in the chondrocytes. The increase of Ihh levels in OA involve not only accelerated pathogenesis but also the initiation of events that are not ordinarily encountered in healthy articular cartilage, perhaps not even as part of the natural aging process. Thus, the level of Ihh in articular cartilage and/or synovial fluid is a biomarker for predicting the disease progression, and Ihh levels over time serve as a biomarker for patients at risk for OA.

Upregulation of Ihh leads to increased hypertrophic markers in cultured OA chondrocytes, including type X collagen and MMPs while inhibition of Ihh expression or activity inhibits the expression of these genes (FIG. 5). Therapeutic methods to inhibit Ihh lead to chondroprotection in patients with early stage disease.

All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### EXAMPLES

The invention now being generally described, it will be more readily understood by reference to the following examples, which are included merely for purposes of illustration of certain aspects and embodiments of the present invention, and are not intended to limit the invention.

### Example 1

### Activation of Indian Hedgehog Promotes Chondrocyte Hypertrophy and Upregulation of MMP-13 in Osteoarthritic Cartilage

### Material and Methods

Knee joint cartilage and SF were obtained during patient OA knee joint replacements (N=36). Normal control samples were obtained from tumor amputations and healthy volunteers (N=5). X-ray demonstrated cartilage damage in OA patients and no joint changes was found in normal controls. Expression of Ihh on the cartilage and in SF were determined by immunohistochemistry and western blot respectively. The density of Ihh expression and the size of chondrocytes were calculated using Ivision software. mRNA levels of Ihh, type X, and MMP-13 were compared between OA cartilage and its adjacent normal cartilage. To determine whether Ihh plays a role in chondrocyte hypertrophy and matrix degradation, mRNA levels of collagen type X and MMP-13 were determined by real time PCR from human OA chondrocytes incubated with Ihh protein and Ihh SiRNA.

Ihh is principally synthesized in the prehypertrophic chondrocytes during growth plate development. It plays a crucial role in regulating the onset of chondrocyte hypertrophy and endochondral bone formation. Recent mouse genetic studies show that Ihh promotes chondrocyte hypertrophy in the developmental growth plate and may play a role in articular chondrocytes. However, direct genetic evidence for Ihh in adult mice and OA patients has not been reported because tissue-specific deletion of Ihh (targeted by Col2al-Cre) mice died shortly after birth. The role of Ihh was examined by comparing the level of Ihh in OA cartilage to normal cartilage and by quantifying collagen type X and MMP-13 after up-regulating or knocking-down Ihh gene expression in human OA chondrocytes.

Expression of Ihh protein was undetectable in normal articular cartilage, but was significantly increased in cartilage of OA patients (FIG. 1). The expression of Ihh was mainly located in the superficial zone of articular cartilage, which was correlated with the severity of OA cartilage damage as determined by modified Mankin score. Western blot analysis indicated that the concentration of Ihh in synovial fluid was much higher in OA patients compared to the age-matched controls (FIG. 2). High levels of mRNA of Ihh, collagen type X, and MMP-13 were found in OA cartilage compared to the adjacent normal cartilage. Furthermore, treatment of OA chondrocytes with Ihh protein resulted in an increase in collagen type X, a marker for chondrocyte hypertrophy and MMP-13, a critical enzyme for cartilage matrix degradation (FIG. 5). Conversely, knockdown of Ihh by transfecting chondrocytes with Ihh SiRNA inhibited the expression of collagen type X and MMP13 in those cells.

OA articular chondrocytes recapitulate the differentiation process that happens during fetal development, which does not occur to an appreciable degree in normal adult articular cartilage. In the developmental growth plate, Ihh is expressed by the prehypertrophic chondrocytes. The expression of Ihh was found to be associated with the severity of OA cartilage damage. The levels of Ihh, collagen type X, and MMP-13 in OA samples were found to be much higher in comparison to normal controls. The results indicate that Ihh regulates the chondrocyte hypertrophic phenotype and cartilage degradation during OA development. Upregulated Ihh signaling was correlated with accelerated chondrocyte hypertrophy as determined by collagen type X and cartilage matrix degradation by MMP-13. Direct evidence from the normal and OA patients indicates that OA cartilage degeneration is accompanied by a response of chondrocytes to this damage which involves enhanced Ihh synthesis. These data indicate that Ihh promote chondrocyte hypertrophy and the pathological progression of OA.

### Example 2

### In Vivo Study of Ihh in an Animal Model of OA

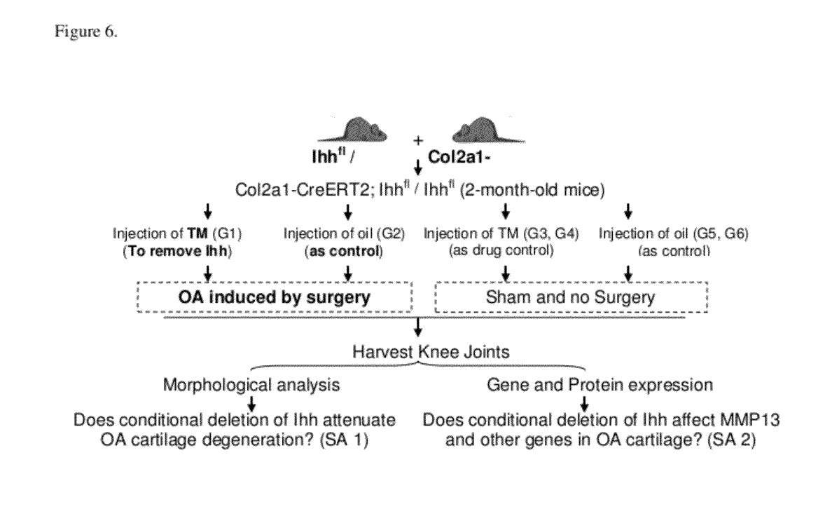

To study the function of Ihh in OA in vivo, an animal model of OA with knee joint instability was used. This mouse model (Col2al-CreERT2; Ihhfl/Ihhfl mice) reproducibly showed cartilage destruction after surgery. The specific design of this study is shown in FIG. 6.

The OA model was induced by knee joint surgery in Col2al-CreERT2; Ihhfl/Ihhfl mice at 2 months of age. The sixty animals were divided into six groups: Tamoxifen (TM) injection+Surgical induced OA (group 1; G1); oil injection+Surgical induced OA (group 2; G2); TM injection+sham surgery (group 3; G3) as surgery control; TM injection+no surgery as TM drug control (group 4; G4); oil injection+sham surgery as surgery control (group 5; G5); oil injection+no surgery as procedure control (group 6; G6). Under general anesthesia, a moderate OA model was created by removing the partial medial menisci (PMM). To activate the tamoxifen-inducible CreERT2 recombinase before the knee joint surgery, mice were injected intraperitoneally with tamoxifen (TM) (sigma) dissolved in oil (sigma) 1 mg/d for 5 consecutive days to remove Ihh at 2-month-old. Two months postsurgery, the animals were euthanized by CO2 and used for the histological analysis.

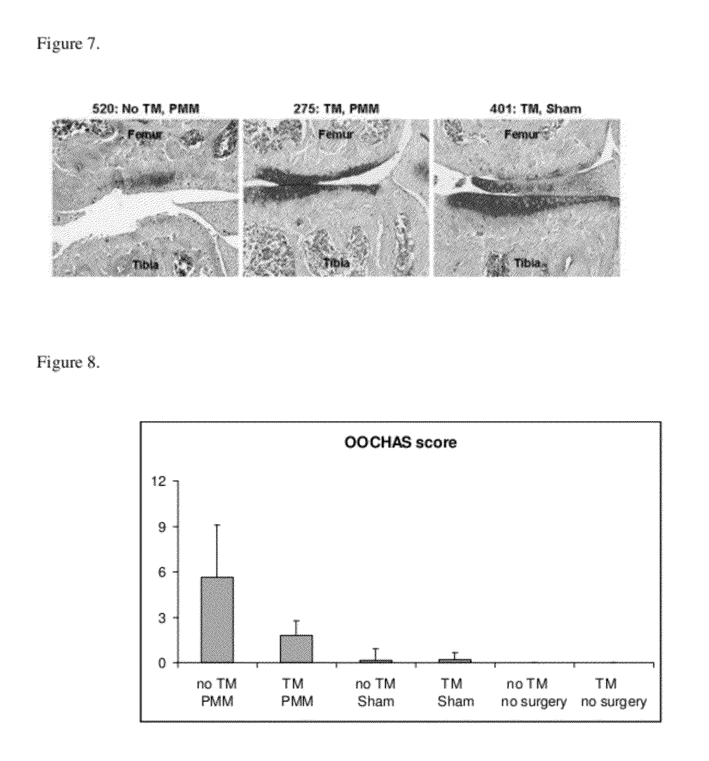

The in vivo data from animal indicates that the deletion of Ihh attenuates OA cartilage damage (middle) in comparison to the no Ihh deletion (left) and sham control (right) (FIG. 7). Morphological changes were quantified using OARSI Score as shown in FIG. 8.

These data indicate that Ihh plays a critical role during OA development. The inhibition of Ihh expression or signaling is therefore useful to inhibit or reduce cartilage damage for the treatment of OA.

What is claimed:

1. A method of diagnosing or predicting the development of osteoarthritis, comprising detecting the presence of an Indian hedgehog (Ihh) gene product in a bodily fluid, wherein an increase a level of said gene product compared to a normal level indicates a diagnosis of osteoarthritis or a prediction of development of osteoarthritis.

2. The method of claim 1, wherein said gene product comprises a polypeptide, protein, or transcript.

3. The method of claim 1, wherein said bodily fluid comprises synovial fluid, blood, or serum.

4. The method of claim 1, wherein said level of said gene product correlates with the severity of osteoarthritis.

5. A method of inhibiting cartilage damage in a subject, comprising identifying an injured or diseased joint, and contacting a tissue of said joint with an inhibitor of an Ihh gene product.

6. The method of claim 5, wherein said tissue is contacted by injecting or infusing said inhibitor into a joint space of said joint.

7. The method of claim 5, wherein said tissue is contacted by injecting or infusing said inhibitor into a synovium of said joint.

8. The method of claim 5, wherein said joint comprises a knee, elbow, hip, or shoulder joint.

9. The method of claim 5, wherein said inhibitor is administered arthroscopically.

10. The method of claim 5, wherein said inhibitor comprises a siRNA.

11. The method of claim 5, wherein said inhibitor is a small molecule inhibitor.

12. The method of claim 11, wherein said small molecule inhibitor is selected from the group consisting of: [chemical structure]


SEQUENCE LISTING (12 sequences)

SEQ ID NO: 1 - DNA, 2074 bp, Homo sapiens
atcagcccaccaggagacctcgcccgccgctcccccgggctccccggccatgtctcccgcccggctccggccccgactgcacttctgcctggtcctgttgctgctgctggtggtgccggcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccccgtcgtggtgtagtcatagagctgcaagctgagctggcgaggggatggttgttgacccctctctcctagagaccttgaggctggcacggcgactcccaactcagcctgctctcactacgagttttcatactctgcctcccccattggggagggcccattccatccatcttaggcccctttgggtgggcttgcgcctcagttgatgctgctaaattccctgggagccagcatggatctggctggacccgatgctgtccagaactgggaaggccacaggggtggggcagccatcccggccattctgaggtatgacattcctccccggccacactcctcaagacacatccagagactgttgctgtctgtgggcagagttctgtgttctggccaatgtgaccgtagtgccggggactgggggaggtgggttggatgtgcttgccacccccccggctaagctcccccttctgctgaaccatgatccccaccccctccgccggtcagtctcccataccttatttattggagtggagggggaagcccatgggagaattttggggatgttttggtcttttcttccttttgtaataaaaattatttaagttgttagagccaaa

SEQ ID NO: 2 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 3 - DNA, 22 bp, Artificial Sequence
  Primer
cattgagacttgactgggcaac

SEQ ID NO: 4 - DNA, 24 bp, Artificial Sequence
  Primer
agagcaggctgagttgggagtcgc

SEQ ID NO: 5 - DNA, 21 bp, Artificial Sequence
  Primer
tgcctcttgtcagtgctaacc

SEQ ID NO: 6 - DNA, 20 bp, Artificial Sequence
  Primer
gcgtgccgttcttatacagg

SEQ ID NO: 7 - DNA, 20 bp, Artificial Sequence
  Primer
tgctgcattctccttcagga

SEQ ID NO: 8 - DNA, 20 bp, Artificial Sequence
  Primer
atgcatccaggggtcctggc

SEQ ID NO: 9 - DNA, 21 bp, Artificial Sequence
  Primer
ctgttcagggacagaatgtgc

SEQ ID NO: 10 - DNA, 20 bp, Artificial Sequence
  Primer
ttggactcacaccatgtgtt

SEQ ID NO: 11 - DNA, 18 bp, Artificial Sequence
  Primer
ccctccagaacctgggac

SEQ ID NO: 12 - DNA, 22 bp, Artificial Sequence
  Primer
aaagaacccaaattcttcaaaa